(19) United States
(12) Patent Application Publication
(10) Pub. No.: US 2019/0223824 A1
(43) Pub. Date: Jul. 25, 2019

(54) RADIOGRAPHIC APPARATUS AND RADIOGRAPHIC SYSTEM

(71) Applicant: Konica Minolta, Inc., Tokyo (JP)

(72) Inventors: Satoshi KOMIYA, Tokyo (JP); Tetsuo Kubota, Tokyo (JP); Takeshi Nukanobu, Tokyo (JP); Masahiro Kuwata, Tokyo (JP)

(21) Appl. No.: 16/235,566

(22) Filed: Dec. 28, 2018

(30) Foreign Application Priority Data
Jan. 24, 2018 (JP) 2018-009642

(51) Int. Cl. A61B 6/00

(52) U.S. Cl. CPC A61B 6/548; G01T 7/00; A61B 6/461; A61B 6/563

(57) ABSTRACT

A radiographic apparatus includes: a hardware processor which generates image data of a radiographic image upon reception of external radiation, is capable of storing the generated image data in a storage, is capable of communicating with an external device via a communication unit, is capable of operating itself in a normal imaging mode or a memory imaging mode, and is capable of concurrently determining for a plurality of types of predetermined conditions whether each condition is satisfied. The hardware processor switches from the normal imaging mode to the memory imaging mode based on occurrence of the determination that at least one of a plurality of first predetermined conditions is satisfied in the normal imaging mode.

### BACKGROUND

### Technological Field

The present invention relates to a radiographic apparatus and a radiographic system.

### Description of the Related Art

Conventionally, for radiography using a radiation apparatus that generates radiation and an imaging apparatus that generates the image data of radiographic images upon reception of radiation, a control device (console) would need to be connected to these apparatuses by wire or wirelessly so that predetermined control signals are transmitted from the console to the apparatuses. In other words, a conventional radiographic system is supposed to include a control device.

However, such a radiographic system supposed to be provided with the above mentioned console cannot conduct imaging, for example, in the event that the console is not actuated or an abnormality occurs in the communication network between the system and the console.

For this reason, in recent years, as disclosed in Japanese Patent Laid-Open No. 2016-214401, for example, an imaging apparatus has been proposed which has a function of operating in either one of the operation mode: the slave mode in which imaging is conducted according to a control signal from a control device and the stand-alone mode in which imaging is conducted by automatically sensing radiation even without reception of a control signal from the control device, and a function of keeping monitoring the status of communication between the control device and the imaging apparatus; and switches the operation mode of the imaging apparatus to the stand-alone mode in the event of the deterioration of the communication status.

Japanese Patent Laid-Open No. 2016-214401 also discloses switching the imaging mode based on occurrence of the operation of a switch provided to the imaging apparatus.

This allows imaging to be continued even in the event that the control device cannot control the imaging apparatus due to the deterioration of the communication status.

However, the imaging apparatus described in Japanese Patent Laid-Open No. 2016-214401 automatically switches the operation mode only according to the status of communication with the control device. For this reason, in imaging with this apparatus, it is possible that the apparatus unintentionally communicates with, for example, a control device placed in the room next to the room where the imaging apparatus conducts imaging and is not used for the imaging, the imaging is conducted in the slave mode instead of the intended mode, which is the stand-alone mode, so that the imaging is wasted.

In addition, to switch the operation mode according to the operation of a switch, the user needs to determine whether or not the conditions are satisfied for imaging in the desired operation mode. It is therefore possible that, in the case of imaging conducted in the slave mode which was selected for the reason that, for example, the control device is present in the vicinity, the imaging may be wasted due to a fail in communication with the control device.

### SUMMARY

An object of the present invention is to enable more reliable switching of the imaging mode of a radiographic apparatus that operates in a normal imaging mode in which image data is generated based on occurrence of reception of a control signal from an external device, or a memory imaging mode in which image data is automatically generated based on occurrence of detection of radiation.

To achieve at least one of the abovementioned objects, according to a first aspect of the present invention, a radiographic apparatus reflecting one aspect of the present invention comprises

a hardware processor which generates image data of a radiographic image upon reception of external radiation, is capable of storing the generated image data in a storage, is capable of communicating with an external device via a communication unit, capable of operating itself in a normal imaging mode in which the image data is generated based on occurrence of reception of a control signal from the external device or a memory imaging mode in which image data is automatically generated based on occurrence of detection of radiation, and is capable of concurrently determining for a plurality of types of predetermined conditions whether each condition is satisfied, wherein

the hardware processor switches its operation mode from the normal imaging mode to the memory imaging mode based on occurrence of the determination that at least one of a plurality of first predetermined conditions is satisfied in the normal imaging mode.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a block diagram showing a schematic configuration of a radiographic system according to an embodiment of the present invention;

FIG. 2 is a perspective view of a radiographic apparatus included in the radiographic system shown in FIG. 1;

FIG. 3 is a block diagram showing the electrical configuration of the radiographic apparatus shown in FIG. 2;

FIG. 4 is a diagram showing an illustrative operating unit included in the radiographic apparatus shown in FIG. 2;

FIG. 5 is a flow chart of a process executed in the radiographic apparatus shown in FIG. 2;

FIG. 6 is a flow chart of a process executed in a radiographic apparatus according to a modification of the embodiment;

FIG. 7A is a diagram for explaining a second predetermined condition for switching the imaging mode of the radiographic apparatus show in FIG. 2;

FIG. 7B is a diagram for explaining a second predetermined condition for switching the imaging mode of the radiographic apparatus show in FIG. 2;

FIG. 8B is a diagram showing an illustrative operating unit included in the radiographic apparatus shown in FIG. 2;

FIG. 9 is a flow chart of a process executed in a radiographic apparatus according to Example 1 of the embodiment;

FIG. 10 is a flow chart of a process executed in a radiographic apparatus according to a modification of Example 1;

FIG. 11A is a diagram showing an illustrative dotted display included in the radiographic apparatus shown in FIG. 2;

FIG. 11B is a diagram showing an illustrative dotted display included in the radiographic apparatus shown in FIG. 2;

FIG. 11C is a diagram showing an illustrative dotted display included in the radiographic apparatus shown in FIG. 2;

FIG. 12 is a flow chart of a process executed in a radiographic apparatus according to Example 4 of the embodiment;

FIG. 13A is a diagram showing an illustrative dotted display included in the radiographic apparatus shown in FIG. 2;

FIG. 13B is a diagram showing an illustrative dotted display included in the radiographic apparatus shown in FIG. 2;

FIG. 13C is a diagram showing an illustrative dotted display included in the radiographic apparatus shown in FIG. 2; and

FIG. 13D is a diagram showing an illustrative dotted display included in the radiographic apparatus shown in FIG. 2.

### DETAILED DESCRIPTION OF EMBODIMENTS

Hereinafter, one or more embodiment of the present invention will be described with reference to the drawings. However, the scope of the invention is not limited to the disclosed embodiments.

### [Configuration of Radiographic System]

First, a description will be given of the schematic configuration of a radiographic system according to this embodiment. FIG. 1 is a block diagram showing the configuration of a radiographic system 100 of this embodiment.

As shown in FIG. 1, the radiographic system 100 of this embodiment includes a radiation apparatus 1, a radiographic apparatus (hereinafter referred to as imaging apparatus 2), and a console 3, and the like.

In addition, the radiographic system 100 is connectable to a radiology information system (RIS), a picture archiving and communication system (PACS), and the like.

The radiation apparatus 1 generates radiation and includes a generator 11, an exposure switch 12, and a radiation source 13, and the like.

The generator 11 is configured to optionally apply a voltage dependent on predetermined radiation irradiating conditions (e.g., tube voltage, tube current, and irradiation time (mAs value)) to the radiation source 13 based on occurrence of the operation of the exposure switch 12.

The radiation source 13 (bulb) includes a rotating anode, a filament, and the like not shown in the drawing. Upon application of a voltage from the generator 11, the filament irradiates the rotating anode with an electron beam dependent on the applied voltage, and the rotating anode generates radiation X (e.g., X rays) with a dose dependent on the intensity of the electron beam.

Although FIG. 1 illustrates the radiation apparatus 1 in which the exposure switch 12 is connected to the generator 11, the exposure switch 12 may be provided to another device (e.g., a master console not shown in the drawing) connected to the generator 11.

In addition, the radiation apparatus 1 may be installed in an imaging room or incorporated in a visiting car or the like to be movable.

The imaging apparatus 2 can communicate with external devices (e.g., the console 3) by wire or wirelessly.

Further, the imaging apparatus 2 can generate the image data of a radiographic image dependent on the external radiation, and transmit it to the console 3.

Note that the details of the imaging apparatus 2 will be described later.

The console 3 is composed of a PC or mobile terminal, or a dedicated device, and can communicate with the radiation apparatus 1, the imaging apparatus 2, and the like by wire or wirelessly.

In addition, the console 3 receives image data from the imaging apparatus 2 by wire or wirelessly, and can perform predetermined image processing on the image data and show a diagnostic image, which is based on the image data, on the display.

Further, the console 3 has a function of associating imaging order information (e.g., examinee information (name and ID), part to be imaged, and imaging method) with the image data received from the imaging apparatus 2.

### [Configuration of Radiographic Imaging Apparatus]

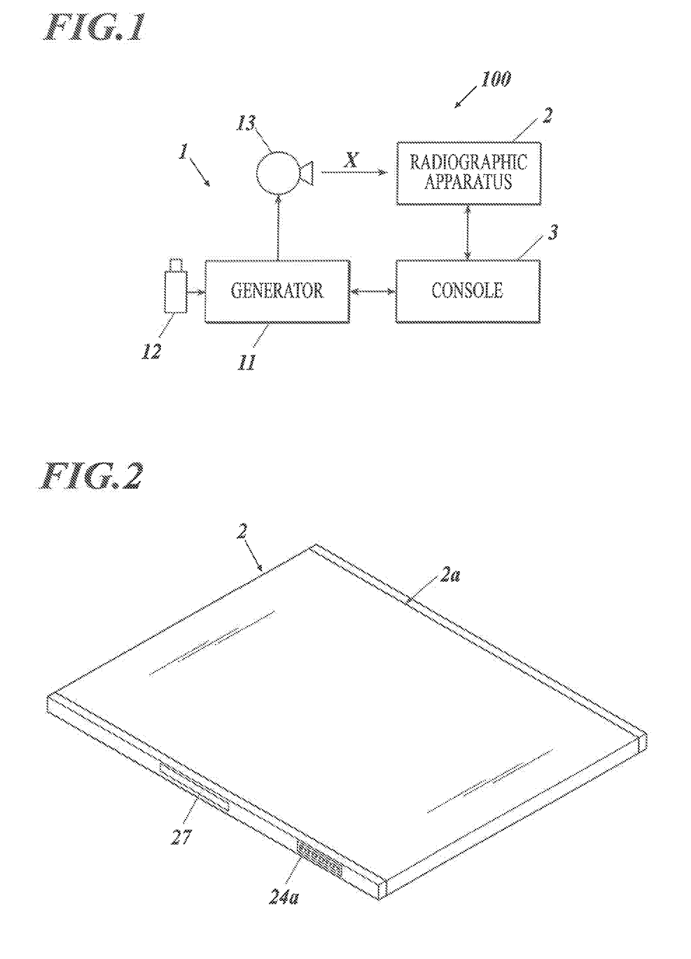

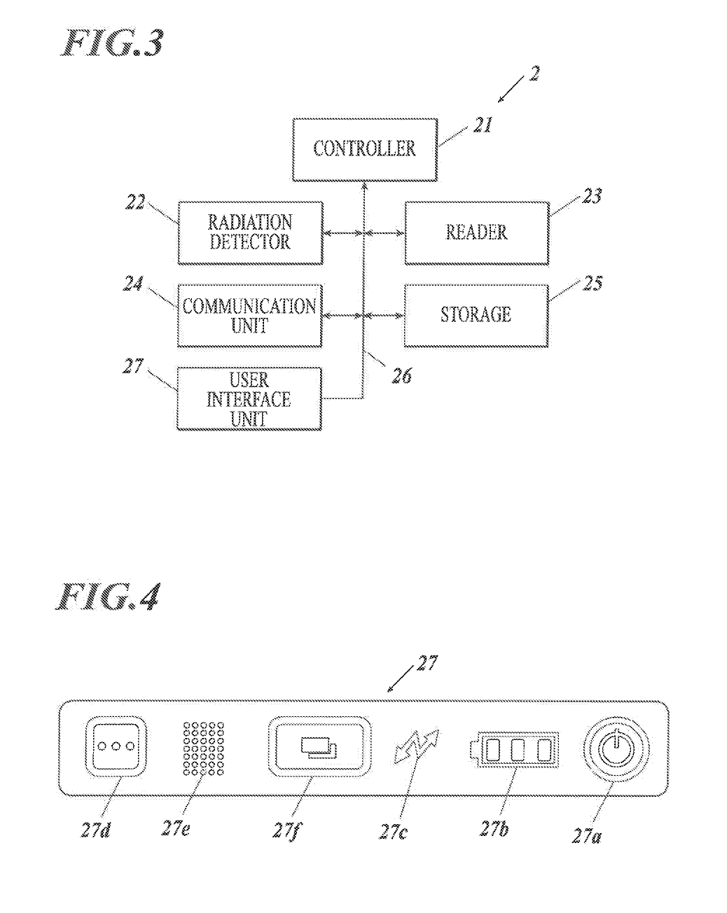

A description will now be given of the details of an imaging apparatus 2 included in the radiographic system 100. FIG. 2 is a perspective view of the imaging apparatus 2, and FIG. 3 is a block diagram showing the electrical configuration of the imaging apparatus 2.

Although FIG. 2 illustrates the transportable panel-like imaging apparatus 2, the present invention is also applicable to fixed-type apparatuses for installation in a room.

As shown in FIG. 2, the imaging apparatus 2 includes a housing 2a. This housing 2a contains, for example, a controller 21, a radiation detector 22, a reader 23, a communication unit 24, a storage 25, and a bus 26 for connection between the components of 21 to 25 which are shown in FIG. 3.

In the case where the imaging apparatus 2 is transportable, it is preferable that a battery (not shown in the drawing) be built in it and electric power be supplied from the battery to the components of 21 to 25.

As shown in FIG. 2, a user interface unit (hereinafter referred to as UI unit 27) is provided on a surface (e.g., side surface) of the housing 2a. As shown in FIG. 4, the UI unit 27 is provided with a power switch 27a, a remaining battery indicator 27b, a communication status indicator 27c, and the like.

The controller 21 is configured to collectively control the operations of the components of the imaging apparatus 2 through a central processing unit (CPU), a random access memory (RAM), and the like. To be specific, for example, when the power switch 27a is turned on, a predetermined control signal is received from the radiation apparatus 1 or console 3, or radiation is received from the radiation apparatus 1, various processing programs stored in the storage 25 are read and expanded in the RAM, and various types of processing are executed according to the processing programs.

The radiation detector 22 may be a component (optionally a known component) having a substrate with a two-dimensional array of a plurality of pixels each including a radiation detector element that generates charge in an amount depending on the dose of radiation directly or indirectly upon reception of external radiation, and a switching element that is provided between each radiation detector element and the wire and enables switching between the on state in which electrical continuity is established between the radiation detector element and the wire and the off state in which the continuity is broken.

In other words, the imaging apparatus 2 may be a so-called indirect-type apparatus that includes a scintillator and senses light generated when the scintillator receives radiation, or a so-called direct-type apparatus that directly senses radiation without a scintillator.

The reader 23 may be a component (optionally a known component) that is configured to optionally read the amount of charge accumulated in the multiple radiation sensor elements as signal values and generate the image data of a radiographic image according to the signal values.

The communication unit 24 is composed of a network interface or the like, and can communicate with external devices (e.g., the console 3) connected to it via a communication network, such as a local area network (LAN), a wide area network (WAN), or the Internet.

The communication unit 24 may include a connector 24a to which a cable for wired communication can be inserted.

The storage 25 is composed of a hard disk drive (HDD), a semiconductor memory, or the like, storing various processing programs including various image processing programs, parameters or files needed to execute the programs, and the like.

The controller 21 of the imaging apparatus 2 with such a configuration has the following functions.

For example, the controller 21 has a function of receiving imaging conditions (e.g., accumulation time, radiation detection sensitivity, and binning) in which imaging is conducted in the memory imaging mode (the details will be described later) from the console 3 via the communication unit 24 when the imaging apparatus 2 is connected to the console 3, and setting the imaging conditions.

Further, the controller 21 has a function of turning off the switching element of the radiation detector 22 so that charge is accumulated in the radiation detector element, and turning on the switching element so that the charge accumulated in the radiation detector element is released to the reader 23.

It also has a function of generating the image data of a radiographic image based on the multiple signal values output from the reader 23.

Further, the controller 21 has a function of operating itself in the normal imaging mode or memory imaging mode.

Here, the “normal imaging mode” refers to an operation mode in which image data is generated based on occurrence of reception of a control signal from an external device (the console 3).

The “memory imaging mode” refers to an imaging mode in which image data is automatically generated based on occurrence of detection of radiation. In the memory imaging mode, automatic transition to the exposure waiting state is performed upon termination of the generation of image data. For this reason, imaging can be repeated even without control by the console 3. In other words, in addition to still image capturing, serial imaging can be achieved in which a series of pieces of image data based on radiation irradiated from the radiation apparatus 1 is repeatedly generated when the imaging apparatus 2 repeats charge accumulation and signal value reading more than once in a short time according to a single imaging operation (pressing the exposure switch 12).

In addition, the controller 21 has a function of concurrently determining for a plurality of types of predetermined conditions whether each condition is satisfied.

Examples of first predetermined conditions include:
Not capable of communicating with the console (the console is remote from it);
Present in an imaging room dedicated to imaging in the memory imaging mode; and
Adjacent to the radiation apparatus with settings for the memory imaging mode.
   The controller 21 determines whether at least one of these conditions is satisfied. The larger the number of first predetermined conditions satisfied, the higher the authenticity of the determination.

Further, the controller 21 has a function of switching the operation mode from the normal imaging mode to the memory imaging mode based on occurrence of the determination that at least one of the first predetermined conditions is satisfied in the normal imaging mode.

To be specific, after the power switch 27a is turned on, the controller 21 first selects the normal imaging mode as the imaging mode as shown in FIG. 5 (Step S1). Subsequently, whether each first predetermined condition is satisfied is determined (Step S2).

Step S2 is repeated until it is determined that at least one of the first predetermined conditions is satisfied. If it is determined that the first predetermined condition is satisfied (Step S2; Yes), the operation mode is switched to the memory imaging mode (Step S3).

After switching from the memory imaging mode to the normal imaging mode, only Steps S2 and S3 are carried out.

The controller 21 also has a function of causing the generated image data to be stored in the storage 25. In this embodiment, image data generated through imaging in the memory imaging mode is stored.

The controller 21 also has a function of transmitting various types of information, signals, or the like including image data to an external device (the console 3) via the communication unit 24 when the imaging apparatus 2 is connected to the console 3.

In this embodiment, an operating unit which is operable by a user may be provided and the imaging mode may be switched when a predetermined operation is performed on the operating unit even when the controller 21 determines that the first predetermined conditions or second predetermined conditions are not satisfied.

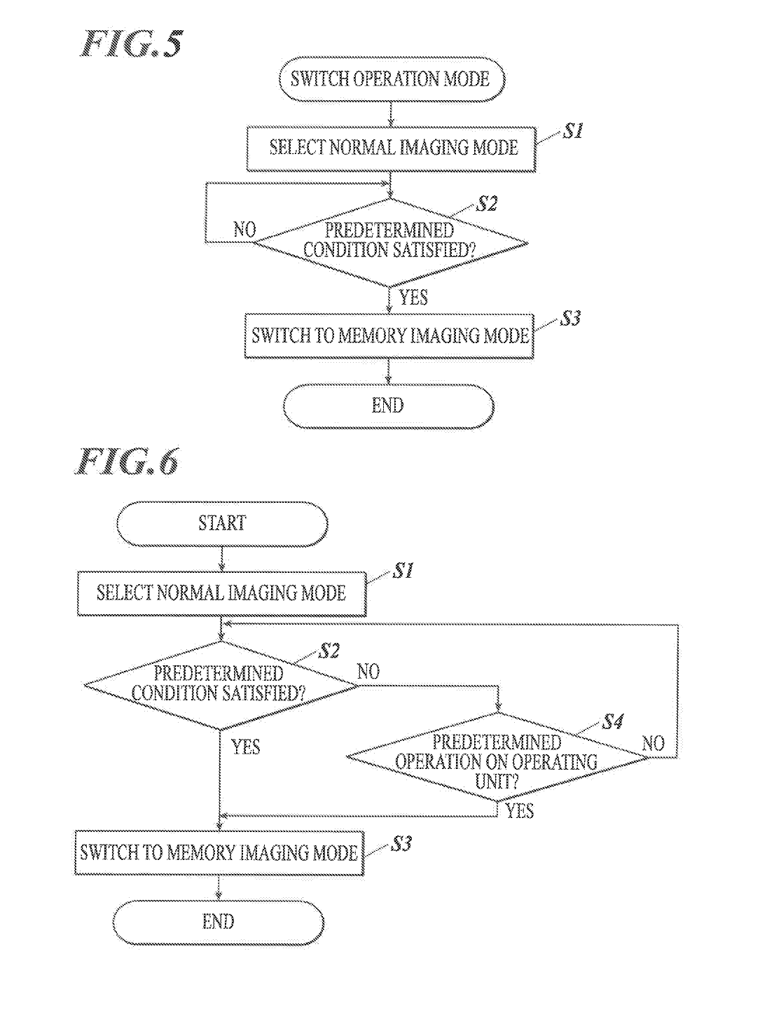

To be specific, the UI unit 27 is provided with a mode switch 27d like that shown in FIG. 4, for example, and the controller 21 is given a function of executing processing like that shown in FIG. 6.

In other words, when it is determined that the predetermined conditions are not satisfied (Step S2; No) in Step S2 described above, whether a predetermined operation (pressing and holding the switch for a predetermined time) is performed on the mode switch 27d is determined (Step S4). When it is determined that the predetermined operation is not performed in Step S4 (Step S4; No), the process returns to Step S2. In contrast, when it is determined that the predetermined operation is performed in Step S4 (Step S4; Yes), the process proceeds to Step S3 (switching to the memory imaging mode).

Accordingly, even when the imaging apparatus 2 is not in environments dedicated to memory imaging, it can conduct imaging in the memory imaging mode.

Further, in this embodiment, the controller 21 preferably has a function of switching the operation mode from the memory imaging mode to the normal imaging mode based on occurrence of the determination that a second predetermined condition other than the first predetermined conditions is satisfied in the memory imaging mode.

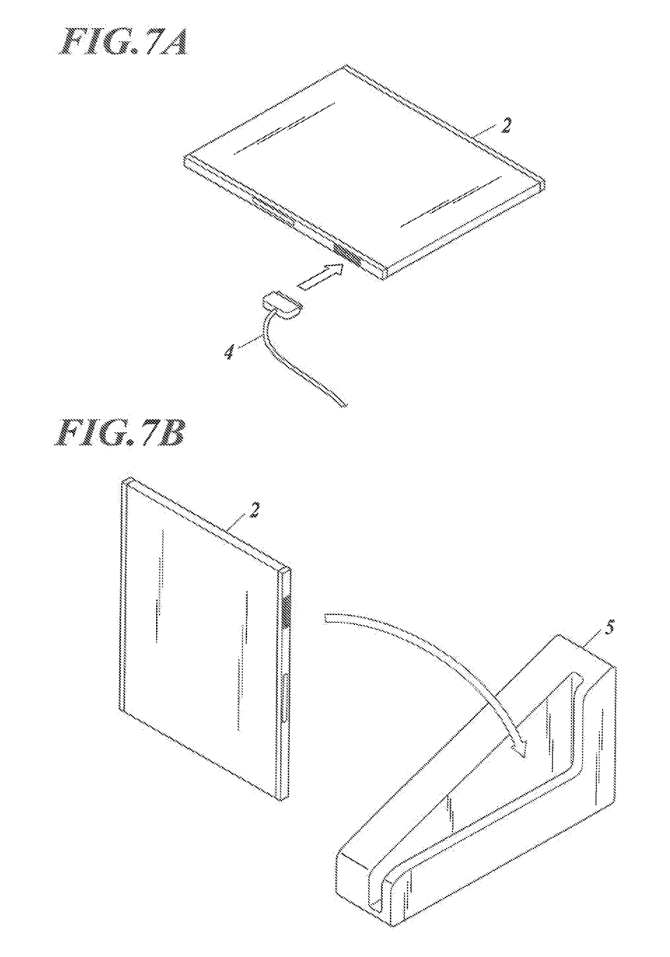

In this case, examples of the second predetermined conditions include
The imaging apparatus 2 is connected to the console 3 by wire (e.g., the cable 4 is connected to the connector 24a as shown in FIG. 7A, or the imaging apparatus 2 is inserted to a cradle 5 as shown in FIG. 7B); and
A predetermined operation (e.g., switching and holding the mode switch 27d (see FIG. 4)) is performed on the UI unit 27.
   Whether any of these is satisfied is determined.

Moreover, in this embodiment, the selected operation mode is preferably notified.

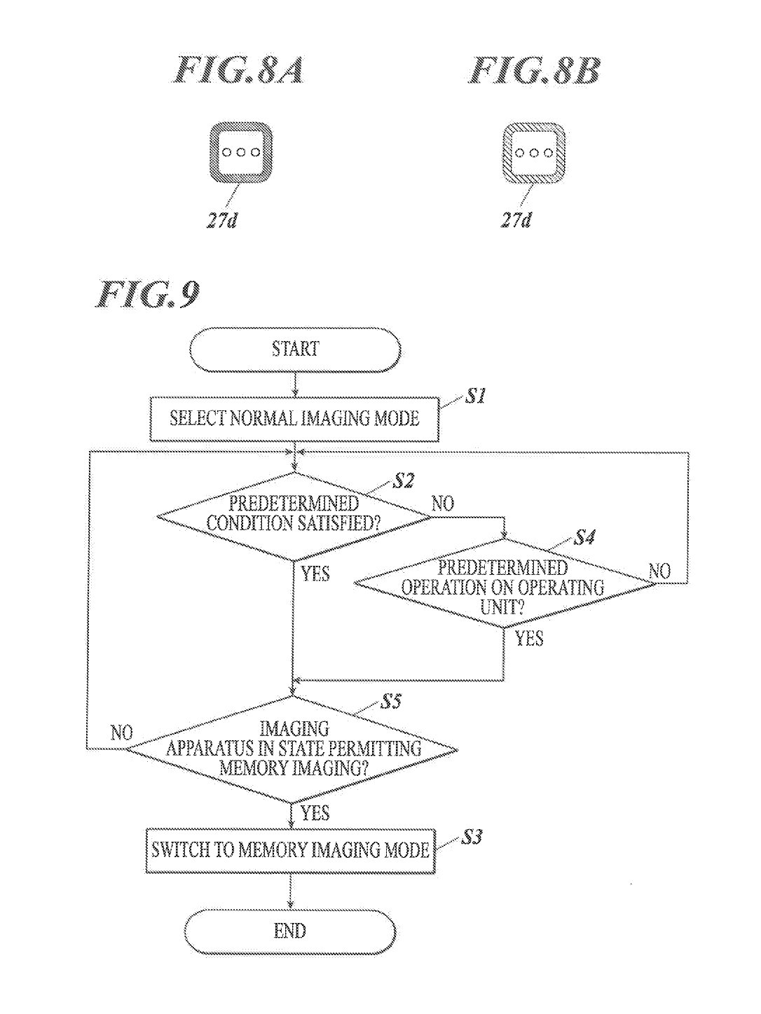

To be specific, as shown in FIGS. 8A and 8B, for example, the color of the mode switch 27d may be changed according to the operation mode.

Thus, the currently selected operation mode can be easily informed.

As described above, the radiographic apparatus 2 according to this embodiment includes a hardware processor (the controller 21) which generates the image data of a radiographic image upon reception of radiation X from the external radiation apparatus 1; is capable of storing the generated image data in the storage 25; is capable of communicating with an external device (the console 3) via the communication unit 24; is capable of operating itself in the normal imaging mode in which image data is generated based on occurrence of reception of a control signal from the external device, or the memory imaging mode in which image data is automatically generated based on occurrence of the detection of radiation X; and is capable of determining for a plurality of types of predetermined conditions whether each condition is satisfied. In the normal imaging mode, the hardware processor switches its operation mode from the normal imaging mode to the memory imaging mode based on occurrence of the determination that at least one of the first predetermined conditions is satisfied.

Thus, satisfaction of each first predetermined condition, which triggers switching to the memory imaging mode, can be concurrently determined, thereby achieving more reliable switching of the imaging mode.

### EXAMPLE

Although the present invention has been described with reference to the embodiment, it is needless to say that the present invention is not limited to the above-described embodiment and modifications can be made as appropriate without departing from the scope of the present invention.

A description will now be given of other problems that may arise in the above-described embodiment and concrete examples for solving the problems.

### Example 1

In the normal imaging mode, even when a condition for switching to the memory imaging mode is satisfied (when it is determined that at least one of the first predetermined conditions in the above-described embodiment is satisfied or when a predetermined operation performed on the operating unit is detected), for example, there may be some kind of problems in the state of the imaging apparatus 2 and memory imaging may be hindered.

To be specific, the problems may arise that the imaging apparatus 2 halts in the middle of memory imaging because the remaining electric power of the built-in battery is little, the generated image data is not saved because the remaining capacity of the storage 25 is little, and a failure occurs in the generated image data because of a damage in the imaging apparatus 2.

To solve such a problem, in the above-described embodiment, if the imaging apparatus 2 has a problem, switching to the memory imaging mode may be restricted even when the conditions for switching to the memory imaging mode are satisfied.

To be specific, the controller 21 is given a function of monitoring the state of each component of the imaging apparatus 2 and a function of performing processing like that shown in FIG. 9, for example.

In other words, when it is determined that a first predetermined condition is satisfied in Step S2 (Step S2; Yes), or when a predetermined operation (pressing and holding the mode switch 27d) performed on the operating unit in Step S4 is detected (Step S4; Yes), whether the imaging apparatus 2 is in the state where memory imaging can be conducted without a problem is determined (Step S5). If it is determined that it is not in the state where memory imaging can be conducted without a problem in Step S5 (Step S5; No), the process returns to Step S2. In contrast, when it is determined that it is in the state where memory imaging can be conducted without a problem in Step S5 (Step S5; Yes), the process proceeds to Step S3 (switching to the memory imaging mode).

Thus, failure of memory imaging due to a problem in the imaging apparatus 2 can be prevented.

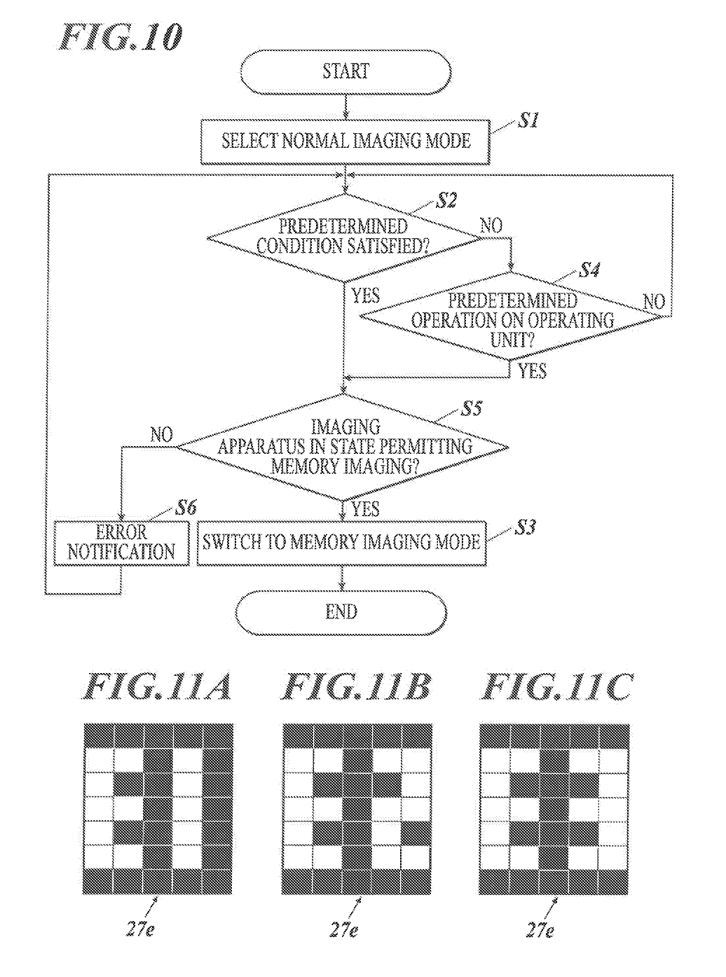

It should be noted that, in Example 1 described above, the UI unit 27 may have a dotted display 27e like that shown in FIG. 4, for example, and the process shown in FIG. 9 may be changed to the process shown in FIG. 10, for example. In other words, when it is determined that it is not in the state where memory imaging can be conducted without a problem in Step S5 (Step S5; No), the error notification that the imaging apparatus 2 has a problem is shown on the dotted display 27e (Step S6).

At the time, the content of the error notification does not simply show a presence or absence of an error but may show a type of error using a number, an alphabet, and the like as shown in FIGS. 11A, 11B, 11C, and 11D, for example.

Thus, the user can be informed of a problem in the imaging apparatus 2, so that failure of memory imaging can be prevented.

### Example 2

It is possible that imaging is conducted in the memory imaging mode even in the state where the console is connected. In such a case, the imaging apparatus 2 generates image data based on occurrence of the sensing of irradiation of radiation and also based on occurrence of reception of a control signal from the console 3, which may hinder memory imaging.

To solve such a problem, in the above-described embodiment, the controller 21 may be given a function of disconnecting communication with the console 3 based on occurrence of switching of the imaging mode to the memory imaging mode.

Thus, memory imaging is conducted only upon the sensing of irradiation of radiation, so that failure of memory imaging can be prevented.

### Example 3

The imaging apparatus is often configured to store image data in a volatile memory because after the generated image data is transferred to the console 3, the image data is no longer needed to be saved.

Meanwhile, in imaging in the memory imaging mode, the imaging apparatus 2 is often not connected to the console 3, and the captured image cannot be checked on the spot but is checked upon later connection to the console.

In memory imaging using an imaging apparatus in which image data is stored in such a volatile memory, the stored image data may be erased after the imaging and before image data is transferred to the console 3, due to, for example, the fact that the imaging switch is turned off or the battery is exhausted.

To solve such a problem, the storage 25 is composed of a volatile memory and a nonvolatile memory in such a manner that the image data generated in the normal imaging mode is stored in the volatile memory, and the image data generated in the memory imaging mode is stored in the nonvolatile memory.

It should be noted that the image data generated in the normal imaging mode and the image data generated in the memory imaging mode may be both stored in the nonvolatile memory.

Thus, the image data generated in the memory imaging mode can be prevented from being erased before connection to the console.

### Example 4

In a typical operation flow of imaging with the imaging apparatus 2 having the memory imaging mode as in the above-described embodiment, the imaging apparatus 2 first performs automatic transition to the exposure waiting state, image data is generated upon irradiation of radiation from the radiation apparatus 1 and is stored in the storage 25, and preparation for the next imaging is carried out.

However, in the case where preparation for the next imaging is carried out after the image data is stored in the storage 25, the problem arises that it takes time to conduct the next imaging.

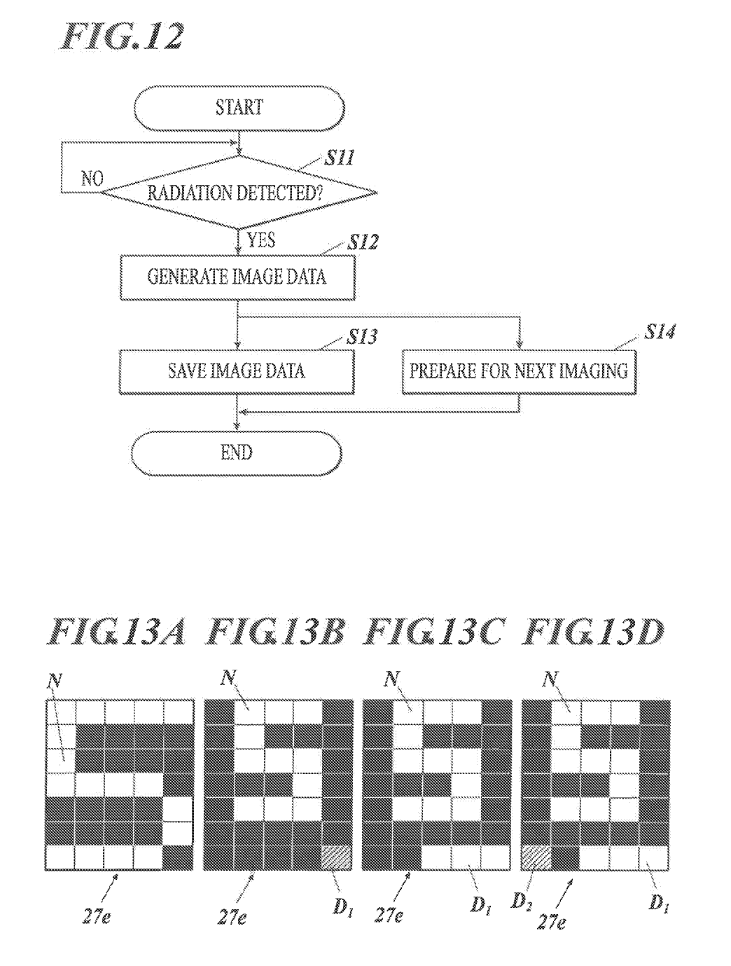

To solve such a problem, in the above-described embodiment, the controller 21 may be given a function of executing the process like that shown in FIG. 12, for example. In other words, after the sensing of irradiation of radiation (Step S11; Yes), image data is generated (Step S12). In addition, image data saving (Step S13) and preparation for the next imaging (Step S14) are concurrently carried out.

Thus, the time to the next imaging can be shortened in the memory imaging mode.

### Example 5

The capacity of the storage 25 of the imaging apparatus 2, i.e., the number of pieces of image data to be stored is limited, which should be noted for imaging. In the normal imaging mode in which the apparatus is connected to the console 3, the number of pieces to be captured can be managed through the console; however, in the case of imaging in the memory imaging mode using the imaging apparatus 2 of the above-described embodiment, the number of pieces to be captured cannot be informed, so that imaging is conducted exceeding the upper limit of the number of pieces of image data to save and the examinee may be wastefully exposed to radiation.

To solve such a problem, the imaging apparatus 2 may be notified of the number of pieces of image captured in the memory imaging mode (the number of files of image data stored in the storage 25).

To be specific, the UI unit 27 is provided with, for example, a dotted display 27e (see FIG. 4) so that the number of captured pieces is represented by a number N as shown in FIG. 13A, for example.

Hence, the user can be easily informed of the number of captured pieces even in the case of imaging conducted in the environment where the console 3 is disconnected or absent.

Since the display area of the dotted display 27e is limited, to express 10 or more pieces (a 2-digit number), as shown in FIGS. 13B, 13C, and 13D, for example, a number corresponding to the ten's place may be represented by the number of dots D₁, which emit light, in a part (e.g., a lower part) of the dotted display 27e. To be specific, FIG. 13B shows the case where 15 pieces are captured, and FIG. 13C shows the case where 35 pieces are captured.

Note that as shown in FIG. 13D, 50 pieces may be represented by a dot D₂ which emits light in a part different from the dots D₁ representing the ten's place. To be specific, together with dot representation of the ten's place, FIG. 13D shows the case where 85 pieces are captured.

### Example 6

An imaging apparatus or console is generally configured to accumulate a log of its operations. In the normal imaging mode, the imaging apparatus 2 and the console 3 are connected to each other, so that a log accumulated in the imaging apparatus and a log accumulated in the console 3 can be associated with each other. Meanwhile, in the memory imaging mode, the imaging apparatus 2 operates independently of the console 3, so that a synchronization between a time based on a log related to the imaging apparatus 2 and a time based on a log related to the console 3 may be lost, for example. Re-connecting the imaging apparatus 2 and the console 3 in such a state may cause a failure in the operation.

To solve such a problem, the storage area for a log in the storage 25 of the imaging apparatus 2 may be divided into two areas so that a log generated in the normal imaging mode and a log generated in the memory imaging mode may be separately accumulated.

Hence, a log can be managed for each imaging mode, minimizing the risk of a failure due to a deviation between a log related to the imaging apparatus 2 and a log related to the console 3 in the memory imaging.

### Example 7

In the above-described embodiment, after imaging conducted in the memory imaging mode, the imaging apparatus 2 is connected to the console 3 and image data saved in the imaging apparatus 2 is transmitted to the console 3. In the memory imaging mode, unlike in the normal imaging mode in which image data is transferred to the console 3 in real time, captured images are collectively transferred later, so that when image data was captured may be unclear.

To solve such a problem, in the above-described embodiment, when the imaging apparatus 2 stores image data in the storage 25, imaging time may be saved together with it.

Hence, for image data generated and transferred to the console in the memory imaging mode, when it was captured can be made obvious.

### Example 8

In the above-described embodiment, after imaging in the memory imaging mode, the imaging apparatus 2 is connected to the console 3, and the image data saved in the imaging apparatus 2 is transmitted to the console 3. In the console 3, received image data is associated with imaging order information. In the memory imaging mode, unlike in the normal imaging mode in which image data is transferred to the console 3 in real time, captured images are collectively transferred later, which may make the association difficult and indefinite.

To solve such a problem, in the above-described embodiment, when the imaging apparatus 2 saves image data, which is obtained by imaging in the memory imaging mode, in the storage 25, additional information may be saved together with it.

Examples of additional information include an image number, imaging time, and imaging conditions.

Hence, association between imaging order information and image data generated and transferred to the console in the memory imaging mode can be made easily and reliably.

### Example 9

An imaging apparatus is generally configured to erase image data stored in the storage 25 after transmission of image data to the console 3. If image data transferred to the console 3 is sequentially erased, image data in the imaging apparatus 2 may be erased although the image data has yet to be transferred to the console 3, for example, because of a poor communication status between the imaging apparatus 2 and the console 3.

To solve such a problem, image data in the storage 25 may be configured to be erased upon termination of transfer of all image data.

To be specific, the controller 21 is given a function of counting the total number of pieces of image data stored in the storage 25, a function of receiving a confirmation signal for the notification that image data has been received from the console, a function of erasing image data in the storage 25 based on occurrence of fact that the number of times a confirmation signal has been received from the console equals the number of layers of image data, and the like.

Hence, image data in the imaging apparatus 2 can be prevented from being erased when the image data has yet to be transferred to the console 3.

### Example 10

Regarding the above-described embodiment, in the case where the imaging apparatus 2 is supposed to be transportable, during transportation of the imaging apparatus 2, a sensor substrate or the like constituting the radiation detector 22, for example, may be damaged due to an accidental drop of the imaging apparatus 2. Since internal damage is hardly sensed, if the imaging apparatus 2 apparently operates, the user may conduct imaging without noticing the damage and a failure may be found in the obtained image data, so that re-imaging may be required.

To solve such a problem, switching to the normal imaging mode may be made upon an impact on the imaging apparatus 2 in the memory imaging mode.

To be specific, the imaging apparatus 2 is provided with an acceleration sensor, a strain gage for sensing the deformation of the housing 2a, and the like, and the controller 21 is given a function of comparing the magnitude of a signal value received from the acceleration sensor or the like with a predetermined threshold and a function of switching to the normal imaging mode upon the determination that the signal value is higher than or equal to the threshold in the memory imaging mode.

Hence, the subject can be prevented from being wastefully exposed to radiation because of imaging conducted by accident with a damage inside the imaging apparatus 2.

### Example 11

Regarding the above-described embodiment, in the case where the imaging apparatus 2 is supposed to be a transportable apparatus that operates on the built-in battery, starting imaging in the memory imaging mode with inadequate remaining battery causes battery exhaustion in the middle and halts the operation of the imaging apparatus 2, so that a desired radiographic image may not be obtained. In that case, re-imaging is required, so that the examinee is wastefully exposed to radiation.

To solve such a problem, when the remaining battery falls less than or equal to a predetermined amount, the mode may be switched from the memory imaging mode to the normal imaging mode.

The console 3 can transmit an instruction to, for example, stop imaging according to the remaining battery of the imaging apparatus 2 to the imaging apparatus 2, thereby preventing a phenomenon in which the battery is exhausted in the middle of imaging, the imaging apparatus 2 halts its operation, and a desired radiographic image cannot be obtained.

### Example 12

In the above-described embodiment, in the case of imaging conducted in the environment where the console 3 is disconnected or absent, the user cannot be informed of the state of the imaging apparatus 2 (the imaging mode or the currently performed operation).

To solve such a problem, the imaging apparatus 2 may notify its state.

To be specific, an operation status display 27f like that shown in FIG. 4 is provided to the UI unit 27 so that the mode (e.g., color) is changed according to the current operation status (during imaging or standby) of the imaging apparatus 2.

Hence, the user can be easily informed of the state of the imaging apparatus 2 even in the case of imaging conducted in the environment where the console is disconnected or absent.

### Example 13

In the above-described embodiment, if imaging mode switching is allowed in any state, during imaging in the memory imaging mode, the button may be pressed and held for some reason and the imaging mode may switch to the normal imaging mode in the middle, wasting the image data that has been obtained by imaging in the memory imaging mode until then, for example.

To solve such a problem, switching to the normal imaging mode may be restricted during imaging in the memory imaging mode.

It should be noted that switching to the memory imaging mode may be restricted during imaging in the normal imaging mode.

This avoids a phenomenon in which the imaging mode switches in the middle and image data that has been obtained by imaging until then is wasted.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-009642, filed on 24 Jan. 2018, is incorporated herein by reference in its entirety.

What is claimed is:

1. A radiographic apparatus comprising:
a hardware processor which generates image data of a radiographic image upon reception of external radiation, is capable of storing the generated image data in a storage, is capable of communicating with an external device via a communication unit, is capable of operating itself in a normal imaging mode in which the image data is generated based on occurrence of reception of a control signal from the external device or in a memory imaging mode in which image data is automatically generated based on occurrence of detection of radiation, and is capable of concurrently determining for a plurality of types of predetermined conditions whether each condition is satisfied, wherein
the hardware processor switches its operation mode from the normal imaging mode to the memory imaging mode based on occurrence of the determination that at least one of a plurality of first predetermined conditions is satisfied in the normal imaging mode.

2. The radiographic apparatus according to claim 1, wherein
the hardware processor switches its operation mode from the memory imaging mode to the normal imaging mode based on occurrence of the determination that at least one of second predetermined conditions other than the first predetermined conditions is satisfied in the memory imaging mode.

3. The radiographic apparatus according to claim 1, further comprising:
an operating unit which is operable by a user, wherein
the hardware processor is capable of switching the imaging mode based on occurrence of a predetermined operation performed on the operating unit even in the event of the determination that the first predetermined conditions or the second predetermined conditions are not satisfied.

4. A radiographic system comprising:
a radiation apparatus which generates radiation;
the radiographic apparatus according to claim 1; and
a console which is capable of receiving image data from the radiographic apparatus by wire or wirelessly.
